(19) United States
(12) Patent Application Publication
(10) Pub. No.: US 2003/0175918 A1
(43) Pub. Date: Sep. 18, 2003

(54) Method for increasing the performance of immobilzed biocatalysts, and catalysts obtained thereby

(76) Inventor: Sobhi Basheer, Sachnin (IL)

Correspondence Address: BANNER & WITCOFF, 1001 G STREET N W, SUITE 1100, WASHINGTON, DC, 20001, US

(21) Appl. No.: 10/204,395

(22) PCT Filed: Feb. 22, 2001
(86) PCT No.: PCT/IL01/00170

(30) Foreign Application Priority Data
Feb. 24, 2000 (IL) 134717

(51) Int. Cl. C12N 11/14; C12N 11/06; C12N 9/20

(52) U.S. Cl. 435/176; 435/181; 435/198

(57) ABSTRACT

A catalyst preparation comprising an insoluble matrix and an enzyme complex immobilized onto said insoluble matrix, characterized in that the matrix contains active carbon. The content of the active carbon is preferably in an amount of 0.1 to 70% by weight, more preferably 1 to 40% by weight and most preferably 3 to 20% by weight, relative to the entire matrix. The enzyme, particularly a lipase, is preferably coated with a surfactant. The inorganic insoluble matrix is preferably a silica-based matrix or an ion-exchange resin. The catalyst preparation of the invention is intended for use as a catalyst in esterification, inter-esterification and trans-esterification reactions.

### FIELD OF THE INVENTION

The present invention relates to an insoluble matrix immobilized biocatalyst, such as a matrix—lipase complex, to a method of preparing same and to the use of same as a biocatalyst.

### BACKGROUND OF THE INVENTION

Enzymatic modification of the structure and composition of oils and fats is of great industrial and clinical interest. This process is accomplished by exploiting regio-specific and non-specific lipases in inter-esterification and/or trans-esterification reactions utilizing fats or oils as substrates (Macrea, A. R., 1983, J. Am. Oil Chem. Soc. 60: 291-294).

Using an enzymatic process, it is possible to incorporate a desired fatty acyl group on a specific position of a triacylglycerol molecule, whereas conventional chemical inter-esterification does not possess regio-specificity. Conventionally, chemical reactions are promoted by sodium metal, sodium alkoxide or cobalt chloride that catalyze acyl migration among triglyceride molecules, leading to the production of triglycerides possessing randomly distributed fatty acyl residues (Erdem-Senatalar, A., Erencek, E. and Erciyes, A. T., 1995, J. Am. Oil Chem. Soc. 72: 891-894).

In recent years, a number of studies have demonstrated the potential application of lipases as promising biocatalysts for different esterification reactions in organic media (Wisdom, R. A., Dunhill, P., and Lilly, M. D., 1987, Biotechnol. Bioeng. 29: 1081-1085).

Many different approaches for the use of lipases in organic media have been attempted in order to activate them and to improve their performance. These include the use of lipase powder suspended in either microaqueous organic solvents or in biphasic systems, and native lipases adsorbed on microporous matrices in fixed- and fluidized-bed reactors (Malcata, et al., 1990, J. Am. Oil Chem. Soc. 890-910). Furthermore, lipases have been hosted in reverse micelles, and in some studies lipases were attached to polyethylene glycol or hydrophobic residues to increase their solubility and dispersibility in organic solvents.

None of the abovementioned approaches was found to be applicable for all enzymatic systems. However, in many cases, when lipases were treated in one way or another as described, their performance with respect to activity, specificity, stability and dispersibility in hydrophobic organic systems was improved.

In recent studies, the development of surfactant-coated lipase preparations has been reported (e.g., Basheer, S., Mogi, K. and Nakajima. M., 1995, Biotechnol. Bioeng. 45: 187-195). This enzyme modification converts slightly active or completely inactive lipases, with respect to esterification of triglycerides and fatty acids in organic media, into highly active biocatalysts. The newly developed surfactant-lipase complexes have been further studied and used for the inter-esterification reaction in organic solvent systems to produce structured triglycerides of major importance in medical applications (Tanaka, Y., Hirano, J. and Funada, T., 1994, J. Am. Oil Chem. Soc. 71: 331-334).

In another approach to the problem, various immobilized-enzyme reactor systems were used in lipase-catalyzed reactions in microaqueous hydrophobic organic media (e.g., Basheer, S., Mogi, K., Nakajima, M., 1995, Process. Biochemistry 30: 531-536). These included fixed- and fluidized-bed reactors, and a slurry reactor. In the published studies, lipase immobilized onto an inorganic matrix was used both in a batch reactor system, and in fixed-bed bioreactor systems. However, the lipases employed were not surfactant-coated and therefore have the same limitations as free lipase systems. These limitations include:

1. Difficulties in recovering the enzyme after completion of the process;

2. Rapid loss of activity of the free enzyme in the reaction medium;

3. Problems of recoverability of expensive enzymes;

4. Low synthetic activity of free lipases in organic solvents.

In a copending patent application of the same applicants hereof (WO99/15689) there is described a dual modification of crude lipase by (1) coating with a surfactant, and (2) immobilization to an insoluble matrix. This procedure results in a synergistic improvement in the efficiency of the enzyme to catalyze trans- and inter-esterification reactions, when compared to either of these two treatments alone. It was also found that it is possible to enhance the catalytic stability of said dually modified lipase for esterification reactions, by providing the enzyme preparation in a granulated form.

Although the above procedures have greatly improved over the prior art results, there still remains a need for improvement of the operational stability of the biocatalyst. Such operational stability is the constancy of efficacy of the catalyst in subsequent batches. The activity of catalysts in general, and biocatalysts in particular, is seldom constant and decreases, often rapidly, when a number of reaction batches are carried out with the same catalyst. This problem is particularly acute with the immobilized lipases discussed above.

It is therefore an object of the invention to provide a method for preparing an immobilized biocatalyst that possesses enhanced operational stability.

It is another object of the invention to provide a method for preparing a biocatalyst exhibiting high activity.

It is a further object of the invention to provide a catalyst that is highly active and that retains its activity when used in subsequent reaction cycles or in continuous reactions, for a long period of time.

It is another purpose of the invention to provide a lipase preparation that possesses the above advantages and that overcomes the prior art disadvantages.

Other objects and advantages of the invention will become apparent as the description proceeds.

### SUMMARY OF THE INVENTION

The invention relates to a catalyst preparation comprising an insoluble matrix and an enzyme complex immobilized onto said insoluble matrix, characterized in that the matrix contains active carbon.

Preferably, but non-limitatively, the active carbon is present in an amount of 0.1 to 70% by weight, and preferably from 1 to 40% by weight and most preferably, 3 to 20% by weight, relative to the entire matrix. This range is that which in most cases, provides the optimal results, but any content that leads to an improvement of the performance of the catalyst is intended to be covered by the present invention.

The invention is not limited to any particular catalyst. However, it has been found that the invention is particularly advantageous when the enzyme used in the catalyst preparation is a lipase. Therefore, lipases are used throughout this specification to exemplify the invention, it being understood that the invention is not limited to any particular enzyme.

When a lipase is employed, its content is preferably 0.1-20 weight percent of the surfactant-coated lipase complex, more preferably 0.01-1.0 weight percent of the entire preparation containing the immobilized matrix.

The lipase can be derived from any suitable source, e.g., from a microorganism such as Burkholderia sp., Candida antractica B, Candida rugosa, Pseudomonas sp., Candida antractica A, Porcine pancreas lipase, Humicola sp., Mucor miehei, Rhizopus javan., Pseudomonas fluor., Candida cylindrcae, Aspergillus niger, Rhizopus oryzae, Mucor javanicus, Rhizopus sp., Rhizopus japonicus and Candida antractica. Alternatively, the lipase can be derived from a multicellular organism.

According to one particular preferred embodiment of the invention the enzyme used in the catalyst preparation is surfactant-coated. An illustrative example of a suitable surfactant is sorbitan monostearate. While, as mentioned above, such coating presents advantages, the invention is by no means limited to any particular treatment or coating of the enzyme. All the advantages of the invention are obtained when using non-coated enzymes.

Many different inorganic insoluble matrices can be used in the practice of the invention. Illustrative and non-limitative examples of such matrices include silica-based matrices and ion-exchange resins. Specific examples of such matrices include, e.g., Celite, Sorbsil, silica powder and Amberlite.

The catalyst preparation of the invention is useful in a variety of reactions. For instance, when the enzyme is a lipase it can be used as a catalyst for esterification, inter-esterification and trans-esterification reactions.

The catalyst preparation of the invention can be provided in any suitable form, one convenient form being the granulated form. Additionally, in some instances it can be desirable to provide an insoluble matrix that has been modified with a fatty acid derivative.

In another aspect the invention is directed to a method for improving the stability of an immobilized enzyme complex, comprising providing a matrix for the immobilization of the enzyme, which matrix contains active carbon.

The examples to follow will illustrate the invention.

### General Procedures

Modified lipases with fatty acid sugar ester surfactants were immobilized on inorganic matrix, such as Celite, silica, calcium sulfate, mixed with different weight ratios of active carbon (charcoal) according to the former procedures in buffer systems. A typical modification and immobilization procedure as follows:

Lipase (300 mg crude containing 7% protein) was dissolved in 100 ml phosphate buffer pH=5.7. Sorbitan monostearate dissolved in ethanol (100 mg/2 ml) was added dropwise to the stirred enzyme solution and then the produced suspension was sonicated for 15 min and magnetically stirred for 2 hours. Inorganic matrix mixed with different weight ratios with active carbon (2 g) was added to the stirred enzyme system and stirred for 4 hours. The produced precipitate was collected by centrifugation or filtration, freeze-dried and the lyophilization over night to remove water. The produced fine powder was used as a biocatalyst or granulated with different binders to produce spheres of 100-1000 μm in diameter.

A list of enzymes used in the above-described procedure is shown in Table I.

TABLE I

| Commercial name | Source | Manufacturer |
|---|---|---|
| Lilipase A-10FG | Rhizopus japonicus | Nagase, Japan |
| Saiken 100 | Rhizopus japonicus | Nagase, Japan |
| Lipase EC | Aspergillus niger | Amano, Japan |
| Lipase AY | Candida rugosa | Amano Japan |
| Lipase LP | Chromobacterium viscosum | Asahi, Japan |
| Lipase PS | Pseudomonas cepacia | Amano, Japan |
| Lipase F-AP15 | Rhizopus oryzae | Amano, Japan |
| Lipase F-EC | Rhizopus oryzae | Extract |
| | | Chemie-Germany |
| Newlase F | Rhizopus niveus | Amano. Japan |
| Lipase G | Penicillium camembertii | Amano, Japan |
| Lipase A | Aspergillus niger | Amano, Japan |
| Lipase M | Mucor javanicus | Roche-Germany |
| Cherazyme Lipase L1 | Burkholderia sp. | Roche-Germany |
| Cherazyme Lipase L2 | Candida Antarctica B sp. | Roche-Germany |
| Cherazyme Lipase L3 | Candida rugosa. sp. | Roche-Germany |
| Cherazyme Lipase L4 | Pseudomonas sp. | Roche-Germany |
| Cherazyme Lipase L5 | Candida Antarctica A. sp. | Roche-Germany |
| Cherazyme Lipase L6 | Pseudomonas sp. | Roche-Germany |
| Cherazyme Lipase L7 | Porcine Pancreas | Roche-Germany |
| Cherazyme Lipase L8 | Humicola sp. | Roche-Germany |
| Cherazyme Lipase L9 | Mucor miehei | Roche-Germany |
| Novozym 388 | Mucor Miehei | Novo nordisk, DK |
| Novozym 525 | Candida Antarctica A. sp. | Novo nordisk. DK |
| Novozym 868 | Candida Antarctica b. sp. | Novo nordisk. DK |

Modified-immobilized Enzyme activity: The activity of modified-immobilized lipases was tested in 1 ml-volume vials by adding 5 mg biocatalyst into n-hexane solution containing 4 mg tripalmitin and 4 mg lauric acid. The vials were incubated at 40° C. for a certain time. Samples were taken periodically, filtered (through 0.45 μm filters) and diluted with a similar volume of acetone and analyzed by GC.

Modified-immobilized Enzyme activity and stability in batch system: The stability of the activated modified and immobilized enzyme on insoluble matrix (powder preparation) was tested in 10 consecutive runs using the same enzyme batch. For this purpose a 1 ml vials containing 1 ml of substrate solution; tripalmitin and lauric acid, each at concentration of 4 mg in 1 ml n-hexane, were mixed with modified-immobilized enzyme powder. The vials were shaken at 40° C. and samples from the reaction mixture were analyzed after 30 min. The immobilized enzyme was left for a few minutes to settle down in order to remove the reaction solution by a syringe and to replace it with another fresh substrate solution. This experiment was repeated 10 times using the same enzyme batch.

Operational Stability of Modified-Immobilized Enzyme

The operational stability of the particulated modified and immobilized enzymes was tested in a jacketed column reactor (0.5 cm i.d. and 15 cm long) using the acidolysis of olive oil (20 mg/ml) and lauric acid (20 mg/ml) in 100 ml n-hexane as a reaction model. The enzyme particles were packed in the column and the substrate solution was recirculated through the packed enzyme (1.5 ml/min). The circulation was stopped after one hour and the reaction solution was analyzed. After each run the solution was discarded and the packed immobilized enzyme was washed with organic solvent (n-hexane) before charging a fresh substrate solution. This procedure was repeated 10 times.

### EXAMPLE 1

### Effect of Matrix on Different Enzymes

The effect of the carbon-containing matrix was tested using enzymes of different origin.

Table II shows the effect of the source of enzyme on initial interesterification reaction rates of tripalmitin (4 mg) and lauric acid (4 mg) dissolved in 1 ml n-hexane. The enzymes were used as crude lipase (A), lipase modified with sorbitan monostearate (SMS) and then immobilized on Celite (B), and lipase modified with sorbitan monostearate (SMS) and then immobilized on Celite containing 1% wt active carbon (C).

TABLE II

| | ri | ri | ri |
|---|---|---|---|
| | (micromol/min. | (micromol/min. | (micromol/min. |
| Type of | mg Biocatalyst | mg Biocatalyst | mg Biocatalyst |
| enzyme | A) | B) | C) |
| Lilipase A-10FG | 0.11 | 8.3 | 15.4 |
| Saiken 100 | 0.10 | 8.9 | 17.2 |
| Lipase EC | 0.1 | 6.7 | 14.6 |
| Lipase AY | 0.0 | 0.2 | 0.6 |
| Lipase LP | 0.1 | 5.8 | 11.4 |
| Lipase PS | 0.0 | 4.7 | 8.94 |
| Lipase F-AP15 | 0.09 | 7.82 | 12.4 |
| Lipase F-EC | 0.07 | 9.7 | 16.5 |
| Newlase F | 0.0 | 0.25 | 0.32 |
| Lipase G | 0.0 | 0.10 | 0.1 |
| Lipase A | 0.0 | 0.12 | 0.1 |
| Lipase M | 0.06 | 6.4 | 10.4 |
| Cherazyme Lipase | 4.24 | 10.24 | 18.3 |
| L1 | | | |
| Cherazyme Lipase | 0.0 | 0.43 | 0.68 |
| L2 | | | |
| Cherazyme Lipase | 0.0 | 0.47 | 0.66 |
| L3 | | | |
| Cherazyme Lipase | 0.24 | 3.7 | 6.4 |
| L4 | | | |
| Cherazyme Lipase | 0.1 | 3.4 | 6.1 |
| L5 | | | |
| Cherazyme Lipase | 011 | 17.6 | 15.4 |
| L6 | | | |
| Cherazyme Lipase | 0.0 | 0.45 | 0.50 |
| L7 | | | |
| Cherazyme Lipase | 0.0 | 1.3 | 2.1 |
| L8 | | | |
| Cherazyme Lipase | 0.26 | 1.4 | 2.4 |
| L9 | | | |
| Novozym 388 | 0.12 | 7.9 | 17.2 |
| Novozym 325 | 0.0 | 0.0 | 0.0 |
| Novozym 868 | 0.0 | 0.45 | 0.54 |

From Table II it can be seen that the addition of carbon to the matrix improves the activity of every catalyst that exhibits an actual initial activity.

### EXAMPLE 2

### Effect of Active Carbon Content

In order to test the effect of active carbon content, a number of catalysts were prepared which differed only in active carbon content. The reaction tested was the interesterification of tripalmitin (4 mg) and lauric acid (4 mg). The catalyst employed was lipaseA-1oFG (5 mg), modified with SMS and immobilized on Celite. The mixture was shaken in 1 ml n-hexane. The results are shown in Table III, in which “ri” is the interesterification reaction rate.

TABLE III

| | | ri |
|---|---|---|
| Active Carbon | | (micromol/min.mg |
| content (%) | Protein content (%) | protein) |
| 0 | 0.308 | 6.7 |
| 0.1 | 0.281 | 10.4 |
| 0.25 | 0.31 | 11.5 |
| 0.5 | 0.32 | 12.4 |
| 0.8 | 0.34 | 12.5 |
| 1.6 | 0.32 | 10.3 |
| 6 | 0.34 | 10.7 |
| 20 | 0.35 | 10.1 |
| 40 | 0.37 | 9.8 |
| 70 | 0.35 | 7.8 |
| 100 | 0.37 | 5.7 |

The results clearly show that the addition of as little as 0.1 wt % of active carbon leads to a dramatic increase in catalyst activity. It should be noted that excessive active carbon content (i.e., above 40 wt %) leads to a decrease in catalyst activity. Whenever a content of 100% active carbon is used, no other immobilized matrix is applied in the preparation.

### EXAMPLE 3

### Enzyme Activity in Batch Systems Using Different Matrices

Four different matrices were tested in an operational stability test. In each test the activity of the activated modified and immobilized enzyme on insoluble matrix was tested in 10 consecutive runs using the same enzyme batch. For this purpose a 1 ml vials containing 1 ml of substrates solution; tripalmitin and lauric acid, each at concentration of 4 mg in 1 ml n-hexane were mixed with modified-immobilized enzyme powder. The vials were shaken at 40° C. and samples were analyzed after 30 min. The immobilized enzyme was left for a few minutes to settle down in order to remove the reaction solution by a syringe and to replace it with another fresh substrate solution. This experiment was repeated 10 times using the same enzyme batch.

### Celite Matrix

Table IV details the conversion (X%) of tripalmitin after 30 min in 10 consecutive runs using the same enzyme batch. Reaction conditions: tripalmitin and lauric acid each 4 mg in 1 ml n-hexane were mixed with 10 mg lipase (Lilipase A-10FG) immobilized on Celite mixed with various weight ratios of active carbon (C). The vials were shaken at 40° C.

TABLE IV

| | X% | X% | X% | X% | X% | X% | X% | X% | X% | X% | X% |
|---|---|---|---|---|---|---|---|---|---|---|---|
| | 0% | (0.1% | (0.25% | (0.5% | (0.8% | (1.6% | (3% | (20% | (40% | (70% | (100% |
| Run | C | C) | C) | C) | C) | C) | C) | C) | C) | C) | C) |
| 1 | 80.9 | 89.0 | 83.4 | 89.8 | 89.0 | 86.2 | 92.1 | 88.5 | 87.8 | 90.1 | 79.6 |
| 2 | 67.0 | 83.8 | 72.3 | 73.9 | 86.9 | 73.4 | 77.3 | 73.2 | 81.8 | 84.3 | 70.4 |
| 3 | 55.9 | 74.6 | 62.6 | 74.0 | 76.0 | 64.6 | 79.2 | 61.0 | 74.5 | 79.2 | 60.5 |
| 4 | 33.7 | 72.7 | 58.0 | 67.3 | 72.1 | 65.8 | 74.0 | 60.6 | 60.2 | 75.2 | 48.3 |
| 5 | 15.4 | 71.3 | 49.7 | 63.2 | 62.8 | 59.9 | 70.1 | 57.6 | 71.2 | 71.0 | 41.6 |
| 6 | 7.0 | 65.4 | 46.8 | 64.2 | 55.9 | 54.0 | 68.6 | 52.8 | 59.6 | 61.8 | 37.8 |
| 7 | 2.1.0 | 47.1 | 36.4 | 63.7 | 55.5 | 47.6 | 66.9 | 47.0 | 47.5 | 60.9 | 31.5 |
| 8 | 0.0 | 43.7 | 28.4 | 57.0 | 52.5 | 45.0 | 61.0 | 45.6 | 59.1 | 54.8 | 28.2 |
| 9 | 0.0 | 25.8 | 27.5 | 46.3 | 50.4 | 43.8 | 59.6 | 40.5 | 55.1 | 57.8 | 27.1 |
| 10 | 0.0 | 17.5 | 27.6 | 42.7 | 47.5 | 41.8 | 57.7 | 30.7 | 52.8 | 50.0 | 25.8 |

### Sorbsil Matrix

Table V details the conversion (X%) of tripalmitin after 30 min in 10 consecutive runs using the same enzyme batch. Reaction conditions: tripalmitin and lauric acid each 4 mg in 1 ml n-hexane were mixed with 10 mg lipase (Lilipase A-10FG) immobilized on Sorbsil (silica) mixed with various weight ratios of active carbon (C). The vials were shaken at 40° C.

TABLE V

| | X% | X% | X% | X% | X% | X% | X% | X% | X% | X% | X% |
|---|---|---|---|---|---|---|---|---|---|---|---|
| | 0% | (0.1% | (0.25% | (0.5% | (0.8% | (1.6% | (3% | (20% | (40% | (70% | (100% |
| Run | C | C) | C) | C) | C) | C) | C) | C) | C) | C) | C) |
| 1 | 20.5 | 91.7 | 90.1 | 89.9 | 86.7 | 89.6 | 93.5 | 87.6 | 81.7 | 83.2 | 79.0 |
| 2 | 18.5 | 88.6 | 86.5 | 79.6 | 64.0 | 77.2 | 65.7 | 77.7 | 70.2 | 66.5 | 70.4 |
| 3 | 14.4 | 85.5 | 79.8 | 76.0 | 59.5 | 74.8 | 79.8 | 73.7 | 65.5 | 58.9 | 60.5 |
| 4 | 11.5 | 84.8 | 82.8 | 72.4 | 63.3 | 71.6 | 80.0 | 71.4 | 64.4 | 55.0 | 48.3 |
| 5 | 10.0 | 82.0 | 83.5 | 64.2 | 62.3 | 70.4 | 75.8 | 72.1 | 57.8 | 38.7 | 41.6 |
| 6 | 8.5 | 82.2 | 81.2 | 65.2 | 58.4 | 66.1 | 74.9 | 66.1 | 53.5 | 32.9 | 37.0 |
| 7 | 7.5 | 81.8 | 76.2 | 65.7 | 54.9 | 67.9 | 70.6 | 69.0 | 53.4 | 37.4 | 31.5 |
| 8 | 2.4 | 79.0 | 75.8 | 61.4 | 52.5 | 61.0 | 67.9 | 63.8 | 51.2 | 26.5 | 28.2 |
| 9 | 2.0 | 74.7 | 67.2 | 56.1 | 51.3 | 60.7 | 64.9 | 62.4 | 49.3 | 25.4 | 27.1 |
| 10 | 1.5 | 77.1 | 66.0 | 56.3 | 50.0 | 61.4 | 64.2 | 63.0 | 40.2 | 21.0 | 25.8 |

### Silica Powder

Table VI details the conversion (X%) of tripalmitin after 30 min in 10 consecutive runs using the same enzyme batch. Reaction conditions: tripalmitin and lauric acid each 4 mg in 1 ml n-hexane were mixed with 10 mg lipase (Lilipase A-10FG) immobilized on Silica powder (Silicon oxide 99%) mixed with various weight ratios of active carbon (C). The vials were shaken at 40° C.

TABLE VI

| | X% | X% | X% | X% | X% | X% | X% | X% | X% | X% | X% |
|---|---|---|---|---|---|---|---|---|---|---|---|
| | 0% | (0.1% | (0.25% | (0.5% | (0.8% | (1.6% | (3% | (20% | (40% | (70% | (100% |
| Run | C | C) | C) | C) | C) | C) | C) | C) | C) | C) | C) |
| 1 | 85.3 | 87.2 | 88.4 | 87.7 | 89.2 | 87.0 | 90.4 | 87.4 | 84.4 | 80.7 | 79.6 |
| 2 | 70.2 | 83.2 | 85.4 | 82.7 | 84.5 | 84.4 | 83.8 | 79.5 | 74.4 | 71.7 | 70.4 |
| 3 | 45.0 | 77.9 | 84.3 | 77.8 | 82.0 | 82.0 | 82.4 | 74.7 | 66.7 | 67.3 | 60.5 |
| 4 | 22.4 | 77.0 | 73.0 | 73.0 | 81.7 | 82.1 | 79.0 | 66.1 | 56.7 | 60.5 | 48.3 |
| 5 | 8.5 | 72.4 | 75.0 | 69.5 | 78.2 | 74.6 | 78.7 | 56.4 | 51.0 | 55.7 | 41.6 |
| 6 | 2.4 | 66.4 | 73.4 | 71.4 | 74.6 | 79.0 | 70.1 | 52.4 | 44.9 | 51.4 | 37.8 |
| 7 | 1.0 | 64.1 | 72.4 | 70.5 | 71.4 | 78.7 | 64.4 | 49.4 | 38.7 | 44.7 | 31.5 |
| 8 | 0 | 49.2 | 64.0 | 65.1 | 69.7 | 75.2 | 60.4 | 40.4 | 33.4 | 37.4 | 28.2 |
| 9 | 0 | 35.0 | 55.2 | 60.5 | 66.0 | 72.0 | 55.0 | 32.2 | 28.5 | 35.4 | 27.1 |
| 10 | 5 | 29.2 | 51.7 | 61.0 | 65.2 | 70.4 | 51.4 | 30.4 | 27.8 | 32.6 | 25.8 |

### Amberlite Matrix

Table VII details the conversion (X%) of tripalmitin after 30 min in 10 consecutive runs using the same enzyme batch. Reaction conditions: tripalmitin and lauric acid each 4 mg in 1 ml n-hexane were mixed with 10 mg lipase (Lilipase A-10FG) immobilized on an ion-exchange resin (Amberlite IR-900) mixed with various weight ratios of active carbon (C). The vials were shaken at 40° C.

TABLE VII

| | X% | X% | X% | X% | X% | X% | X% | X% | X% | X% | X% |
|---|---|---|---|---|---|---|---|---|---|---|---|
| | 0% | (0.1% | (0.25% | (0.5% | (0.8% | (1.6% | (3% | (20% | (40% | (70% | (100% |
| Run | C | C) | C) | C) | C) | C) | C) | C) | C) | C) | C) |
| 1 | 86.4 | 88.7 | 89.3 | 84.7 | 86.4 | 88.1 | 87.0 | 82.4 | 87.6 | 86.4 | 79.6 |
| 2 | 65.0 | 78.7 | 80.8 | 79.6 | 80.4 | 81.7 | 82.5 | 77.9 | 77.0 | 76.8 | 70.4 |
| 3 | 60.4 | 75.4 | 76.0 | 75.6 | 77.9 | 79.4 | 77.0 | 76.3 | 70.8 | 68.0 | 60.5 |
| 4 | 51.0 | 66.3 | 77.3 | 72.7 | 75.0 | 78.7 | 75.1 | 70.1 | 64.7 | 61.4 | 48.3 |
| 5 | 39.7 | 60.1 | 71.6 | 70.0 | 74.9 | 76.8 | 76.4 | 67.8 | 61.3 | 54.6 | 41.6 |
| 6 | 32.3 | 54.0 | 63.5 | 68.9 | 72.0 | 75.4 | 73.0 | 61.7 | 55.8 | 53.1 | 37.8 |
| 7 | 24.0 | 52.9 | 55.7 | 66.8 | 64.7 | 68.7 | 74.3 | 55.4 | 50.0 | 49.7 | 31.5 |
| 8 | 10.9 | 48.6 | 49.0 | 65.2 | 62.9 | 70.4 | 70.1 | 55.6 | 46.4 | 40.0 | 28.2 |
| 9 | 8.1 | 40.0 | 40.7 | 62.0 | 58.0 | 69.4 | 67.0 | 49.4 | 39.4 | 37.7 | 27.1 |
| 10 | 2.4 | 37.4 | 35.0 | 61.3 | 61.1 | 67.2 | 65.3 | 45.7 | 37.2 | 34.7 | 25.8 |

As seen from the above results, the addition of active carbon to the inorganic matrix during the modification and immobilization procedure led in all cases to an increment in the stability of the enzyme.

### EXAMPLE 4

### Effect of Enzyme Load on the Matrix RiceSil-100

Modified lipases with fatty acid sugar ester surfactants were immobilized on RiceSil-100 (a biogenic amorphous Silica) according to former procedures in buffer systems. RiceSil-100 is the commercial name for a silica gel normally used for clarification of oils and fats. This type of silica occurs naturally and contains about 1% wt carbon. A typical modification and immobilization procedure was as follows:

Lipase (300 mg crude containing 7% protein) was dissolved in 100 ml phosphate buffer pH=5.7. Sorbitan monostearate dissolved in ethanol (100 mg/2 ml) was added dropwise to the stirred enzyme solution and then the produced suspension was sonicated for 15 min and magnetically stirred for 2 hours. RiceSil-100 (2 g) was added to the stirred enzyme system and stirred for 4 hours. The produced precipitate was collected by centrifugation or filtration, freeze-dried and the lyophilization over night to remove water. The produced fine powder was used as a biocatalyst or granulated with different binders to produce spheres of 100-1000 μm in diameter.

The above procedure was adopted however the amount of enzyme was varied while the amount of surfactant and that of the matrix were fixed constant. Table VIII shows the interesterification results of Tripalmitin (4 mg) and Lauric acid (4 mg) in 1 ml n-hexane at 40° C. using 10 mg biocatalyst powder. Control reactions were conducted using enzyme immobilized on RiceSil-100 without sorbitan monostearate. Initial reaction rates were defined as ri (micromol/min.mg protein)

TABLE VIII

| | ri - for lipase | ri - for lipase-sorbitan |
|---|---|---|
| Amount of crude lipase | immobilized | monostearate immobilized |
| (g)/2 g matrix | on RiceSil-100 | on RiceSil-100 |
| 0 | 0 | 0 |
| 0.05 | 0.005 | 0.12 |
| 0.15 | 0.12 | 0.42 |
| 0.30 | 0.2 | 1.2 |
| 0.60 | 0.15 | 3.2 |
| 1 | 0.08 | 5.84 |
| 1.5 | 0.07 | 7.55 |
| 2 | 0.07 | 6.2 |
| 3 | 0.06 | 6.1 |
| 4 | 0.05 | 5.7 |
| 6 | 0.03 | 5.4 |

### EXAMPLE 5

### Effect of the Surfactant

Operating as in Example 4, the effect of the surfactant, sorbitan monostearate (SMS) used in the enzyme modification and immobilization technique, on the interesterification activity of tripalmitin and lauric acid, was tested by carrying out runs using different SMS contents. The results are shown in Table IX.

TABLE IX

| | ri - for lipase-sorbitan |
|---|---|
| Amount of SMS mg/mg | monostearate immobilized |
| protein | on RiceSil-100 |
| 0 | 0.005 |
| 1.33 | 0.71 |
| 3.33 | 2.6 |
| 6.66 | 5.6 |
| 13.33 | 6.4 |
| 25.2 | 6.1 |
| 50 | 4.5 |
| 100 | 2.1 |

It can be seen that the addition of SMS generally improves the initial reaction rate, but excessive SMS contents (above 50 mg/mg protein) lead to a lesser improvement, although they still improve over the absence of SMS or low SMS contents.

### EXAMPLE 6

### Operational Stability

The residual interesterification activity of Lilipase a-10FG-sorbitan monostearate complex immobilized on RiceSil-100 was tested in ten consecutive batches using the same biocatalyst Reaction conditions: Tripalmitin (4 mg) and lauric acid (4 mg) dissolved in 1 ml n-hexane at 40° C. The reaction was initiated by adding 10 mg biocatalyst. The reaction system was magnetically stirred for 15 min, the biocatalyst was let to precipitate and then the reaction solvent was removed and replaced with a new fresh reaction solution using the same biocatalyst. This procedure was repeated ten times. The results are shown in Table X, that shows good stability around 60% residual conversion.

TABLE X

| Batch No. | Conversion % |
|---|---|
| 1 | 80 |
| 2 | 72 |
| 3 | 65 |
| 4 | 64 |
| 5 | 63 |
| 6 | 61 |
| 7 | 60 |
| 8 | 58 |
| 9 | 59 |
| 10 | 57 |

### EXAMPLE 7

### Operational Stability with Granulated Catalyst

Example 6 was repeated, using a granulated catalyst. The operational stability of modified Lilipase A-10FG immobilized on RiceSil-100 prepared in buffer pH=5.7 and then granulated with calcium lignosulfate (biocatalyst powder: calcium lignosulfate, 90%:10% (weight)). The activity was expressed as the ratio of the area of the interesterification products and the total area of triglycerides. The results are detailed in Table XI, which shows a further improvement in the residual conversion, over non-granulated catalysts.

TABLE XI

| Batch No. | Conversion % |
|---|---|
| 1 | 72 |
| 2 | 68 |
| 3 | 67 |
| 4 | 67 |
| 5 | 66 |
| 6 | 66 |
| 7 | 65 |
| 8 | 65 |
| 9 | 64 |
| 10 | 65 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A catalyst preparation comprising an insoluble matrix and a surfactant-coated enzyme complex immobilized onto said insoluble matrix, characterized in that the matrix contains from 0.1 to 3% by weight active carbon.

2. The catalyst preparation of claim 1, wherein the active carbon is present in an amount of 0.1, 0.25, 0.5, 0.8, 1.6 or 3% by weight, relative to the entire matrix.

3. The catalyst preparation of claim 1 or 2, wherein the enzyme is a lipase.

4. The catalyst preparation of any one of claims 1 to 3, wherein said matrix is an inorganic insoluble matrix.

5. The catalyst preparation of claim 4, wherein the inorganic insoluble matrix is selected from the group consisting of silica-based matrices and ion-exchange resins.

6. The catalyst preparation of claim 4 or 5, wherein the inorganic insoluble matrix is selected from Celite, Sorbsil and silica powder.

7. The catalyst preparation of any one of claims 1, 2 and 4 to 6, wherein the surfactant is sorbitan monostearate.

8. The catalyst preparation of claim 3, wherein the surfactant is sorbitan monostearate.

9. The catalyst preparation of any one of claims 3 to 8, wherein the content of the lipase is 0.1-20 weight percent of the surfactant-coated lipase complex.

10. The catalyst preparation of any one of claims 3 to 8, wherein the content of the lipase is 0.01-1.0 weight percent of the preparation.

11. The catalyst preparation of any one of claims 3 to 10, wherein the lipase is derived from a microorganism.

12. The catalyst preparation of claim 11, wherein the lipase is derived from a species selected from the group consisting of Burkholderia sp., Candida antractica B, Candida rugosa, Pseudomonas sp., Candida antractica A, Humicola sp., Mucor miehei, Rhizopus javan, Pseudomonas fluor., Candida cylindrcae, Aspergillus niger, Rhizopus oryzae, Mucor javanicus, Rhizopus sp., Rhizopus japonicus and Candida antractica.

13. The catalyst preparation of any one of claims 6 to 8, wherein the matrix is Celite and the active carbon is present in an amount of about 3% by weight, relative to the entire matrix.

14. The catalyst preparation of any one of claims 6 to 8, wherein the matrix is sprbsil and the active carbon is present in an amount of about 3% by weight, relative to the entire matrix.

15. The catalyst preparation of any one of claims 6 to 8, wherein the matrix is Silica powder and the active carbon is present in an amount of about 1.6% by weight, relative to the entire matrix.

16. The catalyst preparation of claim 5, wherein the matrix is Amberlite IR 900 and the active carbon is present in an amount of about 1.6% by weight, relative to the entire matrix.

17. The catalyst preparation of any one of claims 6 to 8, wherein the matrix is RiceSil-100 containing an active carbon in an amount of about 1% by weight, relative to the entire matrix.

18. The catalyst preparation of claim 7 or 8, wherein the sorbitan monostearate is in an amount of 6 to 25 mg per mg protein.

19. The catalyst preparation of any of the claims 13 to 18, wherein the lipase is Lilipase A-10FG.

20. The catalyst preparation of any one of claims 3 to 10, wherein the lipase is derived from a multicellular organism.

21. The catalyst preparation of claim 20, wherein the lipase is porcine pancreas lipase.

22. The catalyst preparation of claim 6, for use as a catalyst for esterification, inter-esterification and trans-esterification reactions.

23. The catalyst preparation of claim 1, wherein said preparation is in granulated form.

24. The catalyst preparation of claim 1, wherein the insoluble matrix has been modified with a fatty acid derivative.

25. A method for improving the stability of an immobilized surfactant-coated enzyme complex, comprising providing a matrix for the immobilization of the surfactant-coated enzyme, which matrix contains from 0.1 to 3% by weight active carbon.

26. The method of claim 25, wherein the catalyst is in granulated form.
